(19) United States
(12) Patent Application Publication
(10) Pub. No.: US 2016/0220251 A1
(43) Pub. Date: Aug. 4, 2016

(54) ADJUSTABLE LOCKING FORCEPS DEVICE FOR THE TREATMENT OF ANTERIOR EPISTAXIS AND METHOD OF USE

(71) Applicant: John Gozar, Jupiter, FL (US)

(72) Inventor: John Gozar, Jupiter, FL (US)

(21) Appl. No.: 14/612,446

(22) Filed: Feb. 3, 2015

(51) Int. Cl. A61B 17/08; A61F 5/08; A61B 17/28; A61B 17/24

(52) U.S. Cl. CPC A61B 17/08; A61B 17/24; A61F 5/08; A61B 17/2833

(57) ABSTRACT

An adjustable locking forceps device for the treatment of anterior epistaxis which is easy to use, adjustable, comfortable for the patient, applies direct and constant pressure to the anatomy, lightweight, compresses both nares simultaneously, compresses the alae and nasal bone, hands free during use, and is anatomically universal. Pressure pads that may be cooled or physically manipulated to yield an endothermic reaction can optionally be attached to the device. This device is believed to be useful in hospitals and clinics wherein quick treatment of epistaxis is required, as well as in the field for use by the lay public and should be included in every first aid kit. The device comprises a barrel hinge bearing assembly having a plurality of barrel sections, a locking means, a right jaw arm and a left jaw arm. Alternate embodiments include compressible pressure pads with varying topographies.

### TECHNICAL FIELD

The present invention is in the technical field of medical devices. More particularly, the present invention is in the technical field of forceps. More particularly, the present invention is in the technical field of adjustable locking forceps for the treatment of anterior epistaxis.

### BACKGROUND OF THE INVENTION

Over a lifetime an individual exhibits an estimated 60% incidence rate of epistaxis thereby ranking nosebleeds as one of the most common ENT emergencies. Epistaxis may arise due to simple abrasion of the inner lining of the nose, or may be related to a much more serious condition such as hypertension. When viewed across a statistically significant sample population, a bimodal age distribution is exhibited wherein the majority of incidents occur between two to ten and fifty to eighty years of age. Of those incidences, 6% of individuals seek medical care for treatment and cessation of local hemorrhaging. Rapid intervention and complication monitoring is indicated for certain high risk groups such as patients of advanced age.

Traditional remedies to treat epistaxis include pinching the nose with the fingers or putting ice over the nares to constrict blood vessels. However, these methods are inconvenient and uncomfortable for patients or medical personnel to hold consistent pressure over the nostrils. In the applicant's experience patients rarely hold the pressure consistently nor long enough for effective use of a manual method. Further, retaining a caregiver for the duration of treatment, which is usually 10 to 15 minutes of consistent pressure, is not feasible in a busy triage environment where attention and resources must be spent on other patients. Cost of retaining a caregiver for this purpose is likewise prohibitive.

In the applicant's experience caregivers have attempted to achieve anterior epistaxis hemostatis by creating a mechanical clamp from tools around the emergency room. Most often the caregiver joins two tongue depressors together to fashion a temporary nose clip; the joining means is usually a rubber band or tied rubber glove. The distal ends of the tongue depressors are then applied like a clamp to the nasal alae while the patient watches the clock. This method is highly undesirable. The wooden tongue depressors used in this method have many disadvantages. They are uncomfortable, may leave splinters in the patient, have thin edges that jab or cut into the patient anatomy, and apply uneven and unreliable pressure according to the manufacturing and individual natural features of the wood. The worst feature of this method is that it is essentially thin wood boards clamped under pressure near to the patient's face and eyes. Should the rubber band or the depressors break, the patient may be injured. Finally, leaving the patient of their own recognizance while wearing the pressured clamp could lead to false claims of breakage and injury and the subsequent claims therefrom.

Other methods and devices include tamponade and thermal or chemical cautery. Although these procedures are also available, in the applicant's experience are rarely used because they are poorly tolerated, unnecessary and increase expenses.

In the lay world, in sports for example, a common treatment technique involves pinching not the nares but the nasal bone with the thumb and forefinger. This method attempts to compress the vessels of the nose against the rigid nasal bone. This method often does not work alone because the sports player does not wait long enough, and has an elevated blood pressure and irregular breathing due to physical activity.

In the applicant's experience there is a deficiency in the existing and prior art wherein there are no adjustable locking forceps device for the treatment of anterior epistaxis which is easy to use, hands free when in place, adjustable, comfortable for the patient, applies direct and constant pressure to the anatomy, lightweight, compresses both nares simultaneously, compresses both the nares and nasal bone, and is anatomically universal.

There is a nose bleed clip disclosed in U.S. Pat. No. 4,457,756. This device is a clip that is inserted into the internal cavities of the bleeding nose. In the applicant's experience this is highly unfavorable and could lead to further injury. Most importantly in the unlikely event that bleeding ceases, the removal of this device is likely to pull any blood that has coagulated onto it while in contact with the clip from the nose and thereby reopening lacerations and causing the bleeding to resume. Further, the disclosure suggests that the pressure of the clip on the nose should be augmented by finger pressure of an individual. Overall this clip does not seem to practically address the cessation of bleeding, nor easy or hands free use during treatment.

There is a nosebleed treatment apparatus disclosed in U.S. Pat. No. 8,303,619 which is essentially a nose clip similar to that used by swimmers to keep water out of the nose during a race. It essentially comprises a u shaped oblong tensing member which is essentially a bendable wire holding two pads. This wire serves to hold the apparatus in place. In the applicant's experience this is also a disfavored means of applying pressure to the nose during treatment because of the lack of tensile strength exhibited by deforming wires. Further, it is suspected that as the nose may move or over time the tensile strength of the wire will lessen thereby causing the bleeding to return. Therefore this apparatus does not seem to successfully address the need for adjustable, fixed, firm, direct and constant pressure during the treatment of epistaxis.

In the applicant's experience, there is a need for an epistaxis treatment device which is i) easy to use, ii) adjustable, iii) comfortable for the patient, iv) applies direct and constant pressure to the anatomy, v) lightweight vi) compresses both nares simultaneously, vii) compresses both the nares and nasal bone, viii) hands free during treatment, and ix) anatomically universal. The device of the present invention is believed to accomplish all of the foregoing objectives.

### SUMMARY OF THE INVENTION

The present invention provides a new and useful adjustable locking forceps device for the treatment of anterior epistaxis which is easy to use, adjustable, comfortable for the patient, applies direct and constant pressure to the anatomy, lightweight, compresses both nares simultaneously, compresses the nares and nasal bone, hands free during treatment, and is anatomically universal. This device is believed to be useful in hospitals and clinics wherein quick treatment of epistaxis is required, as well as in the field for use by the lay public and should be included in every first aid kit. The following description and accompanying drawings disclose at least one version of such a device.

In one of its basic embodiments, the present invention comprises a barrel hinge bearing assembly having a plurality of barrel sections, a locking means, a right jaw arm and a left jaw arm. Alternate embodiments include compressible pressure pads with varying topographies and cross-sections.

When the device is used to treat anterior epistaxis, a patient exhibiting epistaxis is positioned in an upright seated position with head slightly forward. A user, usually a healthcare professional, retrieves an adjustable forceps device according to the present invention, unlocks a locking means of the device to allow the angular rotation of a barrel hinge bearing assembly and increases the angular distance between a right jaw arm and a left jaw arm of the device. The device is then oriented along the face of the patient where the nose of the patient is between the right jaw arm and the left jaw arm of the device and the locking means above the nasal bone and between the eyes of the patient. The user closes the device gently by pushing the left jaw arm and the right jaw arm of the device together, thereby applying mechanically assisted direct, continuous and consistent pressure on the nose of the patient. Once closed on the anatomy of the patient, the user locks the locking means of the device to stop the angular rotation of the barrel hinge bearing assembly and thereby fixes the device on the nose of the patient during treatment. Subsequently the device is maintained in a closed and locked position on the patient's nose for a cessation period of about ten minutes. When epistaxis has ceased, the user unlocks the locking means of the device and removes the device from the patient's nose by widening the angle between the left jaw arm and right jaw arm.

Thus the present invention is believed to provide a new and useful adjustable locking forceps device for the treatment of anterior epistaxis which is easy to use, adjustable, comfortable for the patient, applies direct and constant pressure to the anatomy, lightweight, compresses both nares simultaneously, compresses the alae and nasal bone, hands free during treatment and is anatomically universal. This device is believed to be useful in hospitals and clinics wherein quick treatment of epistaxis is required, as well as in the field for use by the lay public and should be included in every first aid kit. The following description and accompanying drawings disclose at least one version of such a device. Further features and objectives of the present invention will become apparent form the following detailed description and the accompanying drawings.

### BRIEF DESCRIPTION OF THE DRAWINGS

FIG. 1 is an exploded view of an adjustable locking forceps device for the treatment of anterior epistaxis according to the present invention;

FIG. 2 is a perspective view of the device;

FIG. 3 is a right side elevation view of the device;

FIG. 4 is a left side elevation view of the device;

FIG. 5 is an exploded view of an alternate embodiment of the device featuring mechanical teeth and washer;

FIG. 6 is a front elevation view of an alternate embodiment of the device with thick jaw arms;

FIG. 7 is a back elevation view of an alternate embodiment of the device with thick jaw arms; and

FIG. 8 is a view of the device in its environment when in use, locked and applying pressure to the nasal alae, nares and nose bridge of a patient receiving treatment for anterior epistaxis.

### DETAILED DESCRIPTION OF THE INVENTION

As described above, the present invention provides a new and useful adjustable locking forceps device for the treatment of anterior epistaxis which is easy to use, adjustable, comfortable for the patient, applies direct and constant pressure to the anatomy, lightweight, compresses both nares simultaneously, compresses the alae and nasal bone, hands free when during treatment, and is anatomically universal. This device is believed to be useful in hospitals and clinics wherein quick treatment of epistaxis is required, as well as in the field for use by the lay public and should be included in every first aid kit. The following description and accompanying drawings disclose at least one version of such a device.

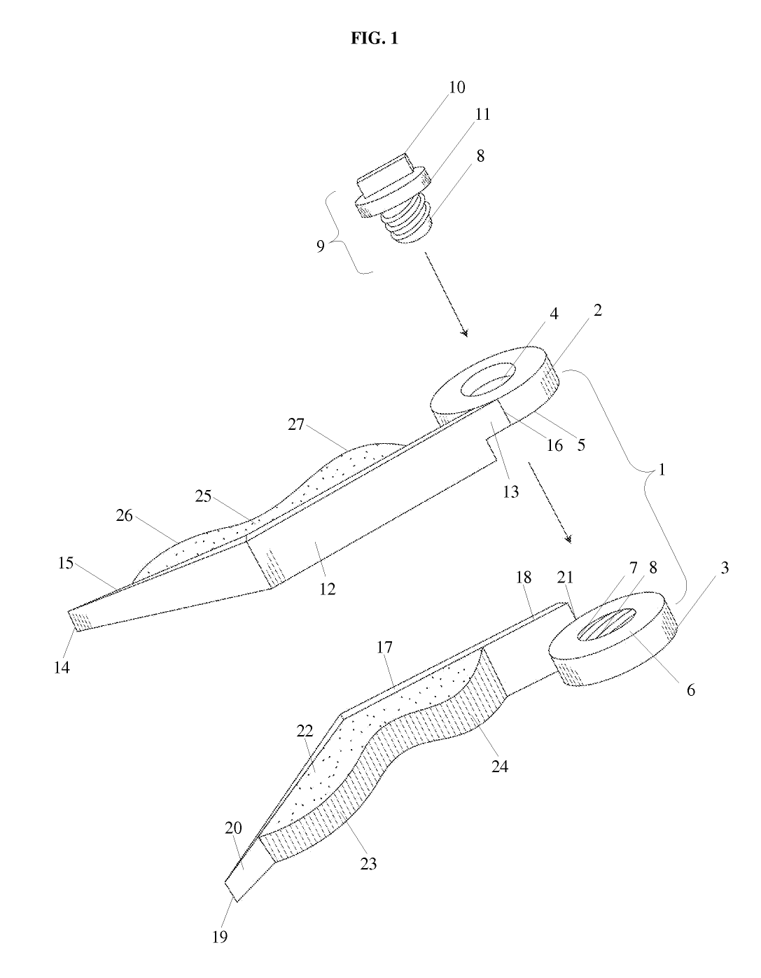

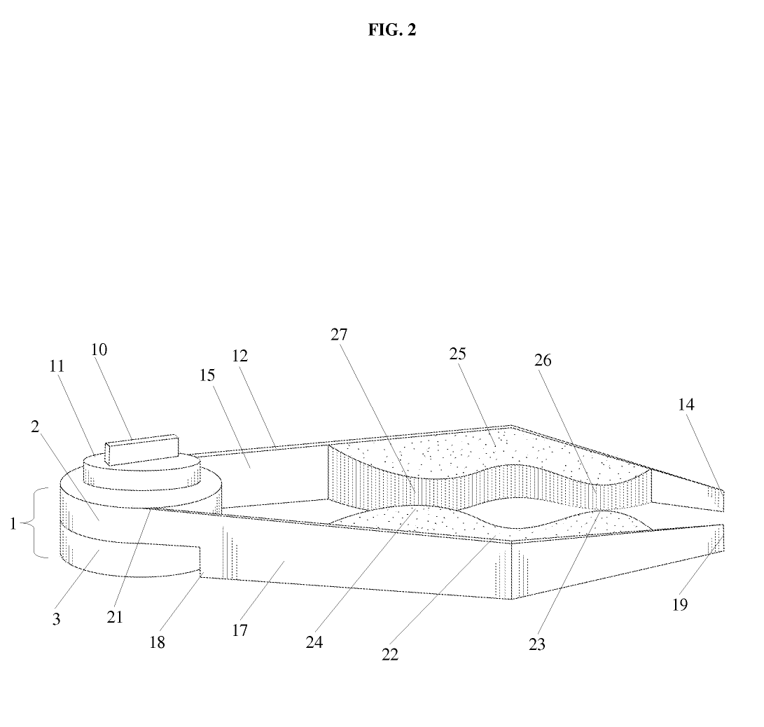

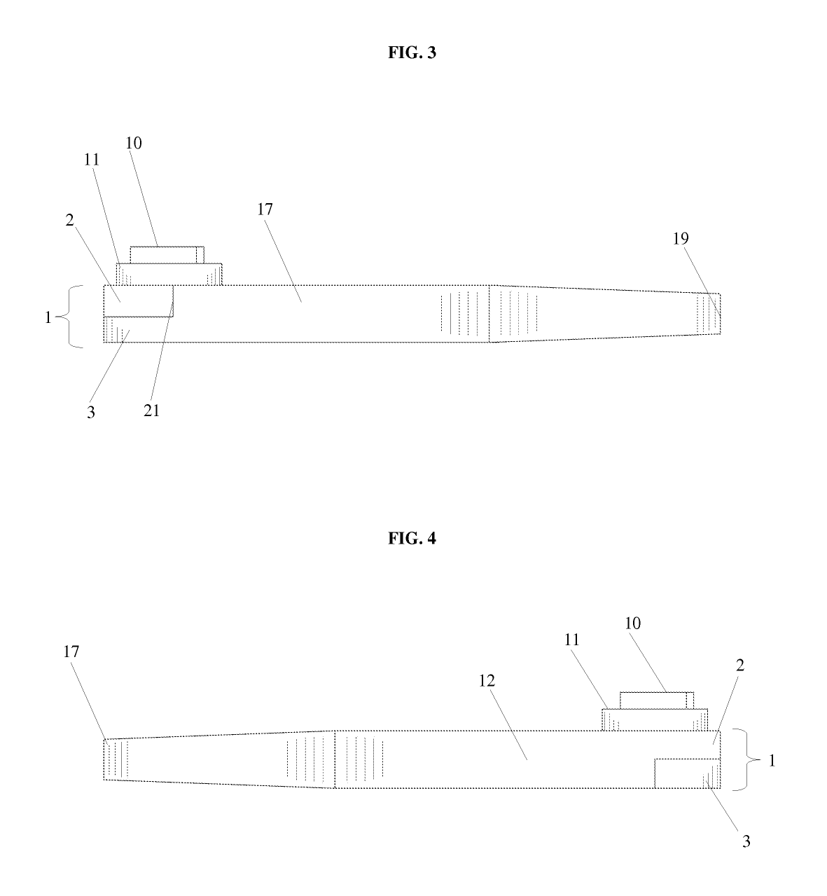

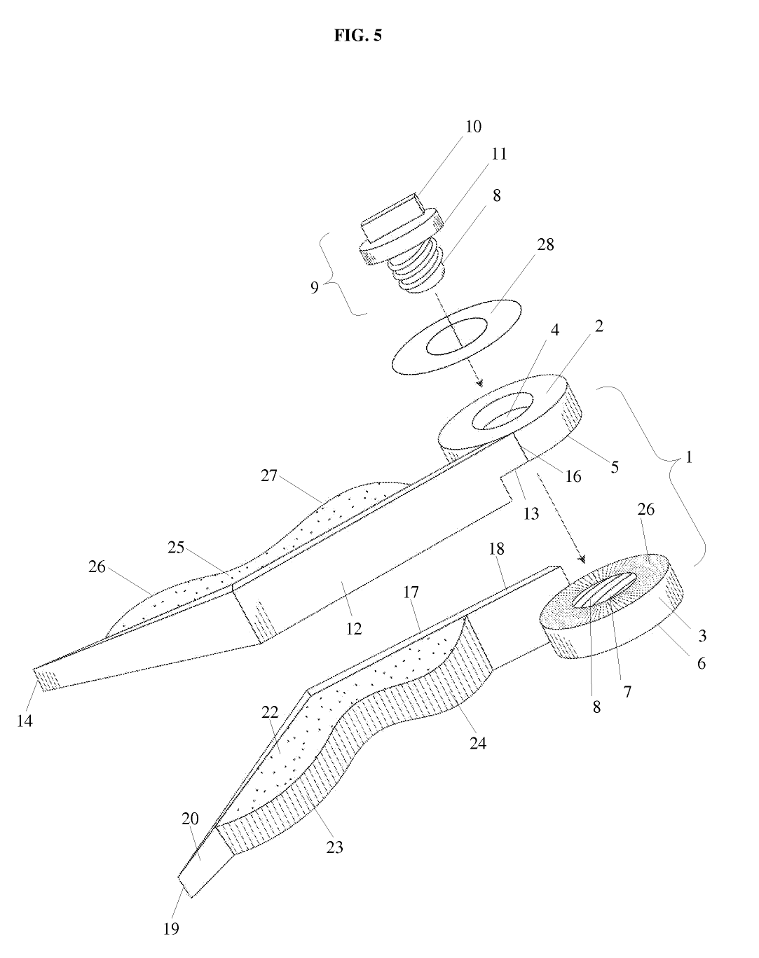

Referring now to the invention in more detail, in FIG. 1 to FIG. 5 there is shown an adjustable locking forceps device for the treatment of anterior epistaxis which comprises a barrel hinge bearing assembly 1 which is generally cylindrical and has an anterior barrel section 2 and a posterior barrel section 3, wherein the barrel hinge bearing assembly 1 allows the anterior barrel section 2 and said posterior barrel section 3 to rotate relative to each other along a fixed central axis of rotation. The anterior barrel section 2 further has an anterior hollow 4 which is a generally cylindrical void that extends completely through a center of the anterior barrel section 2. The void thereof is defined by a perimeter exhibited by the surrounding geometry of the anterior barrel section 2. The anterior barrel section 2 further has a first interior surface 5 which engages with a second interior surface 6 of the adjoining posterior barrel section 3. The posterior barrel section 3 further has a posterior hollow 7 which is a generally cylindrical void extending at least partially through a center of the posterior barrel section 3. The void of the posterior barrel section 3 is defined by a perimeter exhibited by the surrounding geometry of the posterior barrel section 3. The posterior hollow 7 further has a plurality of threads 8 for the receipt of a complementary locking means which is a threaded pivot thumb screw 9, which has at least one manipulating means 10, which are finger grips, knurls or wings, as well as a pressure lip 11 that applies compression forces to the barrel hinge bearing assembly 1 parallel to the fixed central axis of rotation. The pressure lip thereby increases friction forces between the first interior surface 5 and the second interior surface 6 as a user turns said threaded pivot thumb screw 9 thereby locking the device in a fixed orientation when in use.

The device further comprises a left jaw arm 12 which is generally linear in geometry and has a first proximal end 13, a first distal end 14, and a left jaw arm interior surface 15. The first proximal end 13 of the left jaw arm 12 is connected tangent to an exterior perimeter of the anterior barrel section 2 of the barrel hinge bearing assembly 1 along a left arm seam 16.

The device also comprises a right jaw arm 17 which is generally linear in geometry and has a second proximal end 18, a second distal end 19, and a right jaw arm interior surface 20. The second proximal end 18 is connected tangent to an exterior perimeter of the posterior barrel section 3 of the barrel hinge bearing assembly 1 along a right arm seam 21.

A right pressure pad 22 is attached to the right jaw arm interior surface 20 of the right jaw arm 17 and is made of a semi-solid, compressible and deformable material which is silicone or rubber. The right pressure pad 22 has a right nares pressure point 23 which is a raised topographical feature along the right pressure pad 22 for applying pressure to a right nares of a patient when the device is in use. The right pressure pad 22 also has a right nasal bone pressure point 24 which is a raised topographical feature along the right pressure pad 22 for applying pressure to a right nasal bone of a patient when the device is in use.

A left pressure pad 25 is attached to the left jaw arm interior surface 15 of the left jaw arm 12 and is made of a semi-solid, compressible and deformable material which is silicone or rubber. The left pressure pad 25 has a left nares pressure point 26 which is a raised topographical feature along the left pressure pad 25 for applying pressure to a left nares of a patient when the device is in use. The left pressure pad 25 has a left nasal bone pressure point 26 which is a raised topographical feature along the left pressure pad 25 for applying pressure to a left nasal bone of a patient when the device is in use.

In at least one alternate embodiment, the device further comprises a plurality of mechanical teeth 29 arranged radially along the first interior surface 5 and second interior surface 6 for the complementary interaction between the surfaces and allowing the user to fix the device in discreet incremental angles when in use.

In at least one alternate embodiment, the device further comprises a washer 28 between the anterior barrel section 2 and posterior barrel section 3.

In at least one alternate embodiment, the device further comprises a washer 28 between the pressure lip 11 of the threaded pivot thumb screw 9 and the anterior barrel section 2.

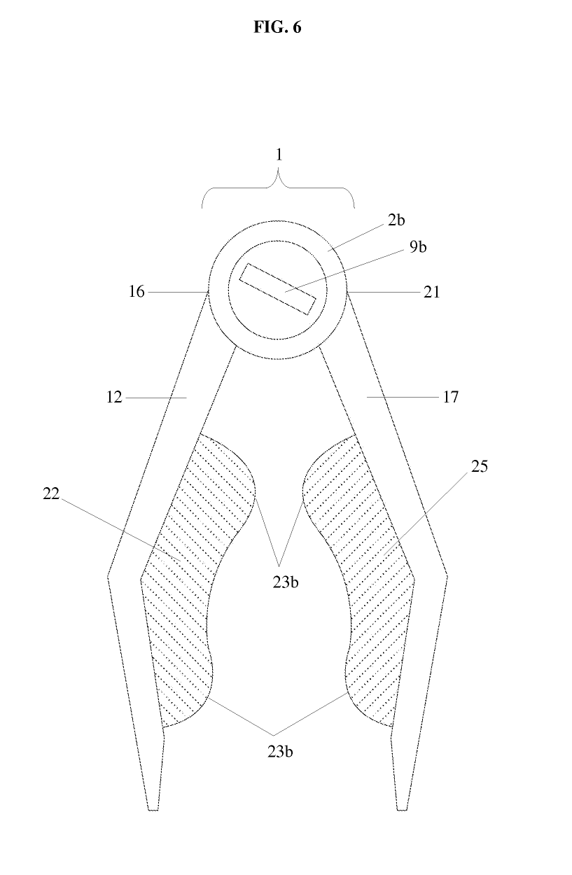

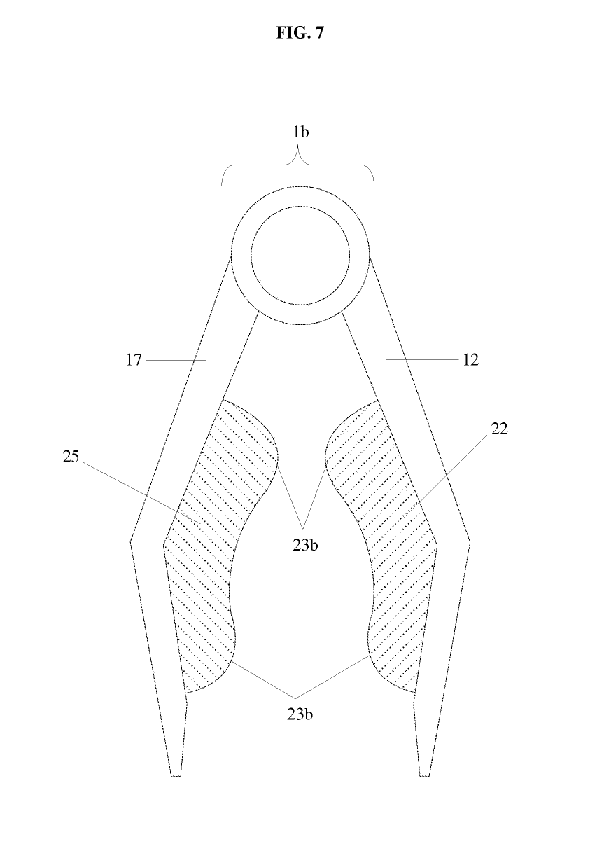

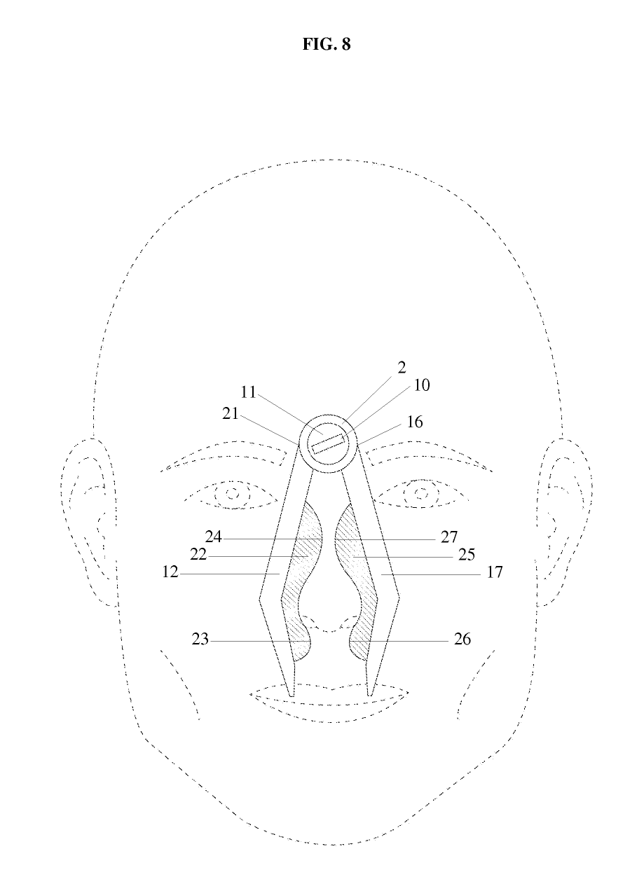

Referring now to the invention, in FIG. 6 to FIG. 8 there are shown alternate embodiments of an adjustable locking forceps device for the treatment of anterior epistaxis. The device comprises a barrel hinge bearing assembly 1 having a plurality of barrel sections 2b, and a locking means 9b attached to the barrel hinge bearing assembly 1. The locking means 9b constrains a right jaw arm 17 which is connected to the barrel hinge bearing assembly 1 along a right arm seam 21. A left jaw arm 12 is connected to the barrel hinge bearing assembly 1 along a left arm seam 17.

In an alternate embodiment of the present invention, the barrel hinge bearing assembly 1 is replace by a hinge means lb, which is a pivot hinge, slip joint or flexible bight.

In an alternate embodiment of the present invention, the device further comprises a right pressure pad 22 attached to the right jaw arm 17. The right pressure pad 22 can be made of any material, but is envisioned that the right pressure pad 22 is made of a semi-solid, compressible and deformable material. It is also envisioned, but not essential to the invention, that the right pressure pad 22 further comprises a plurality of pressure points 23b which are raised topographical features for applying pressure to the anatomy of a patient when the device is in use.

In an alternate embodiment of the present invention, the device further comprises a left pressure pad 25 attached to the left jaw arm 12. The left pressure pad 25 can be made of any material, but it is envisioned that the left pressure pad 25 is made of a semi-solid, compressible and deformable material. It is also envisioned, but not essential to the invention, that the left pressure pad 25 further comprises a plurality of pressure points 23b which are raised topographical features for applying pressure to the anatomy of a patient when the device is in use.

In an alternate embodiment of the present invention, the left pressure pad 25 and right pressure pad 22 further comprise a material that may be cooled and retain lower temperatures to act as thermal vasoconstrictor during use. This includes filling or impregnating the pressure pads with water, fluid or endothermic gel that can be cooled or frozen prior to use. It is also envisioned that chemicals that respond to physical manipulation to yield an endothermic reaction can be utilized. This is often termed and instant cold pack. An instant cold pack is a device that consists of two compartments. One compartment contains water and is near to or inside another compartment containing ammonium nitrate, calcium ammonium nitrate or urea. When the inner compartment of water is broken by squeezing the pack, the water dissolves the solid to yield an endothermic reaction. This reaction absorbs heat from the surroundings, quickly lowering the temperature of the pack. This would be highly desirable for use when the epistaxis device is stored at room temperature, for example in first aid kits. Instant cold packs can also be developed without ammonium nitrate.

In order to treat anterior epistaxis using an adjustable locking forceps device, a patient exhibiting epistaxis is presented, usually in an emergency room. The patient is positioned in an upright seated position with head slightly forward. A user, usually a healthcare professional, retrieves an adjustable forceps device of the present invention. The user unlocks a locking means 9b of the device to allow the angular rotation of a barrel hinge bearing assembly 1 of the device. The user opens the device by increasing the angular distance between a right jaw arm 17 and a left jaw arm 12 of the device. The user orients the device along the face of the patient where the nose of the patient is between the right jaw arm 17 and left jaw arm 12 of the device and the locking means 9b is positioned above the nasal bone and between the eyes of the patient. The user closes the device gently by pushing the left jaw arm 12 and right jaw arm 17 of the device together, thereby applying mechanically assisted direct, continuous and consistent pressure on the nose of the patient. Once closed on the anatomy of the patient, the user locks the locking means 9b of the device to stop the angular rotation of the barrel hinge bearing assembly 1, and thereby fixes the device on the nose of the patient during treatment. Subsequently the device is maintained in a closed and locked position on the patient's nose for a cessation period of about 10 minutes.

When complete and epistaxis has ceased, the user unlocks the locking means 9b of the device and removes the device from the patient's nose by widening the angle between the left jaw arm 12 and right jaw arm 17.

In at least one alternate method of use for an adjustable locking forceps device for the treatment of anterior epistaxis, a topical vasoconstrictor may be sprayed into the nose of the patient during treatment. The user may also elect to clear the nasal vestibule of the patient and apply a cold compress to the nose and cheeks of the patient to promote vasoconstriction during treatment.

The construction details of the invention as shown in FIG. 1 to FIG. 7 are that the device may be made of plastic or any other rigid, strong material, which can include high grade carbon steel which can withstand an autoclave or metal.

In further detail, still referring to the invention of FIG. 1 to FIG. 7 the dimensions of the device can be any size but it is envisioned that at least one embodiment is sufficiently wide and long to comfortably fit a patient nose when in use.

The previously described versions of the present invention have many advantages, including and without limitation, the properties of being i) easy to use, ii) adjustable, iii) comfortable for the patient, iv) applies direct and constant pressure to the anatomy, v) lightweight vi) compresses both nares simultaneously, vii) compresses the alae and nasal bone, viii) hands free during treatment, and ix) anatomically universal. The device of the present invention is believed to accomplish all of the foregoing objectives. The invention does not require that all the advantageous features and all the advantages need to be incorporated into every embodiment of the invention.

Although the present invention has been described in considerable detail with reference to certain preferred versions thereof, other versions are possible. Therefore the spirit and scope of the appended claims should not be limited to the description of the preferred versions contained therein.

The reader's attention is directed to all papers and documents which are filed concurrently with this specification and which are open to public inspection with this specification, and the contents of all such papers and documents are incorporated herein by reference.

All the features disclosed in this specification may be replaced by alternative features serving the same, equivalent or similar purpose, unless expressly stated otherwise. Thus, unless expressly stated otherwise, each feature disclosed is one example only of a generic series of equivalent or similar features.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. As for “means for” elements, the applicant intends to encompass within the language any structure presently existing or developed in the future that performs the same function. The invention should therefore not be limited by the above described embodiment, method, and examples, but by all embodiments and methods within the scope and spirit of the invention.

What is claimed is:

1. An adjustable locking forceps device for the treatment of anterior epistaxis which comprises
a. a barrel hinge bearing assembly which is generally cylindrical and having an anterior barrel section and a posterior barrel section,
said barrel hinge bearing assembly allowing said anterior barrel section and said posterior barrel section to rotate relative to each other along a fixed central axis of rotation,
said anterior barrel section further having an anterior hollow which is a generally cylindrical void extending completely through a center of and defined by a perimeter exhibited by the surrounding geometry of the anterior barrel section,
said anterior barrel section further having a first interior surface which engages with a second interior surface of said posterior barrel section;
said posterior barrel section further having a posterior hollow which is a generally cylindrical void extending at least partially through a center of and defined by a perimeter exhibited by the surrounding geometry of the posterior barrel section,
said posterior hollow further having a plurality of threads for the receipt of a complementary locking means which is a threaded pivot thumb screw,
said threaded pivot thumb screw having at least one manipulating means, which are finger grips, knurls or wings,
said threaded pivot thumb screw further having a pressure lip that applies compression forces to the barrel hinge bearing assembly parallel to the fixed central axis of rotation thereby increasing friction forces between the first interior surface and the second interior surface as a user turns said threaded pivot thumb screw thereby locking the device in a fixed orientation when in use;
b. a left jaw arm which is generally linear in geometry and having a first proximal end, a first distal end, and a left jaw arm interior surface,
said first proximal end connected tangent to an exterior perimeter of the anterior barrel section of the barrel hinge bearing assembly along a left arm seam;
c. a right jaw arm which is generally linear in geometry and having a second proximal end, a second distal end, and a right jaw arm interior surface,
said second proximal end connected tangent to an exterior perimeter of the posterior barrel section of the barrel hinge bearing assembly along a right arm seam;
d. a right pressure pad attached to the right jaw arm interior surface of the right jaw arm,
said right pressure pad made of a semi-solid, compressible and deformable material which is silicone or rubber,
said right pressure pad having a right nares pressure point which is a raised topographical feature along the right pressure pad for applying pressure to a right nares of a patient when the device is in use,
said right pressure pad having a right nasal bone pressure point which is a raised topographical feature along the right pressure pad for applying pressure to a right nasal bone of a patient when the device is in use; and
e. a left pressure pad attached to the left jaw arm interior surface of the left jaw arm, said left pressure pad made of a semi-solid, compressible and deformable material which is silicone or rubber,
said left pressure pad having a left nares pressure point which is a raised topographical feature along the left pressure pad for applying pressure to a left nares of a patient when the device is in use,
said left pressure pad having a left nasal bone pressure point which is a raised topographical feature along the left pressure pad for applying pressure to a left nasal bone of a patient when the device is in use.

2. An adjustable locking forceps device for the treatment of anterior epistaxis which comprises
a. a barrel hinge bearing assembly having a plurality of barrel sections;
b. a locking means attached to the barrel hinge bearing assembly to constrain the
c. a right jaw arm connected to the barrel hinge bearing assembly along a right arm seam; and
d. a left jaw arm connected to the barrel hinge bearing assembly along a left arm seam.

3. The device as in claim 1 further comprising a plurality of mechanical teeth arranged radially along the first interior surface and second interior surface for the complementary interaction between the surfaces and allowing the user to fix the device in discreet incremental angles when in use.

4. The device as in claim 1 further comprising a washer between the anterior barrel section and posterior barrel section.

5. The device as in claim 1 further comprising a washer between the pressure lip of the threaded pivot thumb screw and the anterior barrel section.

6. The device as in claim 2 where the barrel hinge bearing assembly is replace by a hinge means, wherein in the hinge means is a pivot hinge, slip joint or flexible bight.

7. The device as in claim 2 further comprising a right pressure pad attached to the right jaw arm.

8. The device as in claim 2 further comprising a left pressure pad attached to the left jaw arm.

9. The device as in claim 3 wherein the right pressure pad is made of a semi-solid, compressible and deformable material.

10. The device as in claim 3 wherein the right pressure pad further comprises a plurality of pressure points which are raised topographical features for applying pressure to the anatomy of a patient when the device is in use.

11. The device as in claim 4 wherein said left pressure pad is made of a semi-solid, compressible and deformable material.

12. The device as in claim 4 the left pressure pad further comprises a plurality of pressure points which are raised topographical features for applying pressure to the anatomy of a patient when the device is in use.

13. A method of treating anterior epistaxis using an adjustable locking forceps device which comprises:
a. presenting a patient exhibiting epistaxis;
b. positioning the patient in an upright seated position with head slightly forward;
c. retrieving an adjustable forceps device;
d. unlocking a locking means of the device to allow the angular rotation of a barrel hinge bearing assembly of the device;
e. opening the device by increasing the angular distance between a right jaw arm and a left jaw arm of the device;
f. orienting the device along the face of the patient where the nose of the patient is between the right jaw arm and left jaw arm of the device and the locking means is positioned above the nasal bone and between the eyes of the patient;
g. closing the device gently by pushing the left jaw arm and right jaw arm of the device together, thereby applying mechanically assisted direct, continuous and consistent pressure on the nose of the patient;
h. locking the locking means of the device to stop the angular rotation of the barrel hinge bearing assembly, thereby fixing the device on the nose of the patient during treatment;
i. maintaining the device in a closed and locked position on the patients nose for a cessation period of about 10 minutes.
j. unlocking the locking means of the device;
k. removing the device from the patient's nose by widening the angle between the left jaw arm and right jaw arm once the epistaxis has ceased.

14. The method of claim 12 further comprising spraying a topical vasoconstrictor into the nose of the patient.

15. The method of claim 12 further comprising clearing the nasal vestibule.

16. The method of claim 12 further comprising applying a cold compress to the nose and cheeks to promote vasoconstriction during treatment.

17. The invention of claim 2 wherein the left jaw arm and right jaw arm are angled or curved to follow the anatomy of a patient.

18. The invention of claim 2 wherein the device is made from a rigid material, which is plastic, high grade carbon steel or metal.

19. The method of claim 12 further comprising cooling at least one pressure pad on the device.

20. The device as in claim 1 or 2 wherein the left pressure pad and right pressure pad further comprise a materials that can be cooled or physically manipulated to yield an endothermic reaction to act as thermal vasoconstrictor during use.
